(19) United States
(12) Patent Application Publication
(10) Pub. No.: US 2025/0313520 A1
(43) Pub. Date: Oct. 9, 2025

(54) PROCESS AND PLANT FOR PRODUCING RENEWABLE FUELS

(71) Applicant: Topsoe A/S

(72) Inventor: Johannes QUINTERO, Valby (DK)

(73) Assignee: Topsoe A/S, Kgs. Lyngby (DK)

(21) Appl. No.: 18/864,366

(22) PCT Filed: May 8, 2023
(86) PCT No.: PCT/EP2023/062128

§ 371 Date: Nov. 8, 2024

(30) Foreign Application Priority Data
May 11, 2022 (DK) PA202200444

(51) Int. Cl. C07C 29/151; C01B 3/12; C25B 3/07; C25B 13/07

(52) U.S. Cl. CPC C07C 29/1518; C01B 3/12; C25B 3/07; C25B 13/07; C01B 2203/0283

(57) ABSTRACT

Process and plant for producing methanol, the process comprising the steps of: a) providing a raw synthesis gas stream; b) water gas shifting at least a portion of the raw synthesis gas stream, thereby producing a shifted synthesis gas; c) preparing a separate hydrogen containing stream and a separate oxygen containing stream by electrolysis of a water feedstock; d) introducing at least a portion of the separate hydrogen containing stream into shifted synthesis gas, thereby producing a methanol synthesis gas; and e) converting the methanol synthesis gas into said methanol.

The present invention relates to the production of renewable fuels, such as methanol, from a synthesis gas (syngas) prepared by incorporating the electrolysis of a water feedstock for producing hydrogen.

Typically, in a thermal decomposition of renewable feeds, such as gasification for producing methanol syngas, inerts like N₂ and CH₄ are generated and thus present in the syngas. To further utilize the syngas, typically also the syngas is subjected to a cleaning or purification step for cleaning the gas from impurities e.g. heavy metals, silica, etc., and thus cleaned syngas is subjected to a shifting step i.e. water gas shift step (WGS step) for changing the composition of the syngas according to the reaction CO+H₂O=CO₂+H₂. The WGS step may either be sweet (without sulfur in the syngas) our sour (including sulfur in the syngas). Finally, some of the CO₂ is removed in a CO₂ removal section. This results in venting CO₂ from the process. For adjusting of the so-called module M=(H₂−CO₂)/(CO+CO₂) in the syngas to the desired level of about 2.0 for downstream methanol synthesis, typically part of the syngas bypasses the WGS step and the CO₂ removal.

US 2009235587 AA discloses a method and system for producing syngas utilizing heat from thermochemical conversion of a carbonaceous fuel to support decomposition of at least one of water and carbon dioxide using one or more solid-oxide electrolysis cells. Simultaneous decomposition of carbon dioxide and water or steam by one or more solid-oxide electrolysis cells may be employed to produce hydrogen and carbon monoxide. A portion of oxygen produced from at least one of water and carbon dioxide using one or more solid-oxide electrolysis cells is fed in a gasifier or combustor to oxidize the carbonaceous fuel to control the carbon dioxide to carbon monoxide ratio produced.

US 20090289227 A1 discloses a method for utilizing CO₂ waste comprising recovering carbon dioxide from an industrial process that produces a waste stream comprising carbon dioxide in an amount greater than an amount of carbon dioxide present in starting materials for the industrial process. The method further includes producing hydrogen using a renewable energy resource and producing a hydrocarbon material utilizing the produced hydrogen and the recovered carbon dioxide. The carbon dioxide may be converted to CO by electrolysis and water to hydrogen by electrolysis.

WO11134705 A1 discloses plant for producing chemical raw materials or fuels, comprising a gasifier and an apparatus for synthesis from carbon monoxide and hydrogen which is connected to the gasifier, wherein an electrolyser is connected to the apparatus for synthesis from carbon monoxide and hydrogen in order to supply hydrogen.

DE102010027474 A1 discloses a method comprising preparing crude gas obtained by gasification of a fossil fuel into a synthesis gas. A molar ratio from hydrogen to carbon monoxide is set depending on a type of synthesizing selection, where the treatment of the raw gas comprises allowance of hydrogen debited to a water gas shift reaction. The raw gas is obtained from an energy source selected from artificial natural gas, methanol, dimethyl ether or synthetic fuel.

Ali et al., Renewable Energy 154 (2020) 1025-1034, disclose a combined system for methanol production combining autothermal reforming and electrolysis in a solid oxide electrolysis cell (SOEC).

Applicant's US2020109051 discloses a method for the preparation of synthesis gas combining electrolysis of water, tubular steam reforming and autothermal reforming of a hydrocarbon feed stock.

Applicant's co-pending pending patent application WO PCT/EP2021/086999 discloses a method and a system for producing a synthesis gas from a carbon dioxide-rich stream and a water feedstock, where the synthesis gas is further converted to methanol by methanol synthesis.

US 20160319381 discloses a method for reducing CO₂ emissions in the operation of a metallurgical plant which comprises at least one blast furnace for producing crude iron and a converter steel mill for producing crude steel.

WO 2020058859 discloses a process for the production of methanol from gaseous hydrocarbons, such as natural gas, associated petroleum gas, fuel-gas produced in a refinery or in certain chemical plants, or biogas.

It has now been found that by introducing electrolysis technology and integrating this into the process for producing methanol synthesis gas in a particular manner, as recited farther below, it is now possible to remove the need of CO₂ removal and rather utilize CO₂, which allows to increase the overall production of the renewable fuel, e.g. methanol, by up to 40%.

Accordingly, in a first aspect of the invention, there is provided a process for producing methanol, comprising the steps of:
a) providing a raw synthesis gas stream;
b) water gas shifting (WGS) at least a portion of the raw synthesis gas stream, thereby producing a shifted synthesis gas;
c) preparing a separate hydrogen containing stream and a separate oxygen containing stream by electrolysis of a water feedstock, i.e. water and/or steam;
d) introducing at least a portion of the separate hydrogen containing stream into the shifted synthesis gas, thereby producing a methanol synthesis gas; wherein the methanol synthesis gas has a module M=(H₂−CO₂)/(CO+CO₂) in the range 1.80-2.40, such as 1.95-2.10, and a molar ratio CO/CO₂ greater than 2, such as 10 or higher; and
e) converting the methanol synthesis gas into said methanol, i.e. methanol conversion of the shifted synthesis gas;

As used herein, the term “first aspect of the invention” means a process (method) according to the invention. The term “second aspect of the invention” means a plant (system) according to the invention.

As used herein, the term “process/plant” means process or plant.

The term “present invention” or “invention” may be used interchangeably with the term “present application” or “application”, respectively.

As used herein, the term “comprising” may also include “comprising only”, i.e. “consisting only of”.

Hence, in an embodiment there is also provided a process for producing methanol, consisting of the steps:
providing a raw synthesis gas stream;
water gas shifting (WGS) at least a portion of the raw synthesis gas stream thereby producing a shifted synthesis gas;
preparing a separate hydrogen containing stream and a separate oxygen containing stream by electrolysis of water feedstock, i.e. water and/or steam;
introducing at least a portion of the separate hydrogen containing stream into the shifted synthesis gas, thereby producing a methanol synthesis gas; wherein the methanol synthesis gas has a module M=(H₂−CO₂)/(CO+CO₂) in the range 1.80-2.40, such as 1.95-2.10, and a molar ratio CO/CO₂ greater than 2, such as 10 or higher; and
converting the methanol synthesis gas into said methanol.

The step of CO₂-removal and thereby a CO₂-removal section, such as an amine absorber, is thereby obviated. The CO₂-removal section conveys normally a significant capital expense (CAPEX) and operating expense (CAPEX). Again, the available CO₂ is instead utilized for enabling the increase in the overall production of the renewable fuel e.g. methanol, by up to 40%.

The invention provides therefore the advantages of no CO₂-emissions thereby reducing or eliminating the carbon footprint of the process and plant, no need for expensive CO₂-removal technology, such as an amine absorber, after the water gas shifting, and not least a simpler front-end section prior to methanol synthesis.

As used herein, the term “renewable fuel” is used interchangeably with the term “e-fuel” (electro-fuel) and signifies a fuel product in which at least the hydrogen required is provided by electrolysis of a water feedstock. The methanol produced by the present invention may thus be regarded as an e-fuel, and thereby denoted as e-methanol. Suitably, the electrolysis is powered by electricity from renewable sources, such as wind or solar, optionally from thermonuclear power.

By the invention the methanol synthesis gas has a module M=(H₂−CO₂)/(CO+CO₂) in the range 1.80-2.40, such as 1.95-2.10, and a molar ratio CO/CO₂ greater than 2, such as 10 or higher. The synthesis gas used for methanol production is normally described in terms of said module M, since the methanol synthesis gas is in balance for the methanol reaction when M=2. It would be understood that M=(H₂−CO₂)/(CO+CO₂) is calculated in term of molar percentages (molar concentrations). In the methanol synthesis gas there may be some excess of hydrogen resulting in modules slightly above 2, for instance 2.05 or 2.10. The provision of much higher content of CO with respect of CO₂, with the molar ratio CO/CO₂ being greater than 2, such as a ratio of 10 or higher, enables that the methanol reaction proceeds with low generation of water which is detrimental for the methanol synthesis catalyst in the subsequent methanol conversion step, since the methanol synthesis is conducted mainly according to the reaction: CO+2H₂=CH₃OH, rather than typically via the reaction 3H₂+CO₂=CH₃OH+H₂O. The resulting water has also a negative effect on the performance of the catalyst and the catalyst volume increases with more than 100% if the CO₂ concentration is too high, e.g. 90%. Much more energy is also required for the purification of the methanol because all the water is removed by distillation.

In an embodiment, a portion of the raw synthesis gas bypasses the water gas shifting and is then combined with the shifted synthesis gas and the at least a portion of the separate hydrogen containing stream. Hence, the separate hydrogen containing stream is mixed with the shifted syngas and un-shifted gas (raw synthesis gas stream), thereby producing a mixture having the required module M.

This enables increased flexibility and control of the amount of hydrogen in the methanol synthesis gas, within the required range of the module M. It may often be desirable to have a value of M slightly greater than 2, such as 2.05 or 2.10, by having an excess of hydrogen. In other instances, it may be desirable to have M slightly below 2, such as 1.95. The WGS produces hydrogen by the reaction: CO+H₂O=CO₂+H₂ in a stable manner, whereas the separate preparation of hydrogen by electrolysis is intermittent, in particular when the power required for electrolysis is provided from renewable sources such as wind, solar or hydropower. Where, for instance, there is no much wind and thereby less production of hydrogen by electrolysis, the amount of bypassed raw synthesis gas is reduced. Where, for instance, there are windy conditions and thereby there is a high production of hydrogen by electrolysis, the amount of bypassed raw synthesis gas is increased.

In an embodiment, the process further comprises a cleaning step for providing the raw synthesis gas, i.e. the raw synthesis gas has been subjected to a cleaning step, thereby removing impurities. The cleaning is for instance conducted in a syngas purification section, whereby under the addition of e.g. water, impurities which may be detrimental for downstream steps are removed.

In an embodiment, the process further comprises, prior to said step a), a thermal decomposition of a renewable feed stream for producing a crude synthesis gas stream, and subsequently subjecting the crude synthesis gas stream to said cleaning step, e.g. in a syngas purification section, for removing impurities, thereby producing the raw synthesis gas stream.

As used herein, the term “thermal decomposition” means any decomposition process, in which a material is partially decomposed at elevated temperature, typically 250° C. to 800° C. or perhaps 1000° C., in the presence of sub-stoichiometric amount of oxygen (including no oxygen). The product will typically be a combined liquid and gaseous stream, as well as an amount of solid char. The term shall be construed to included processes known as gasification, pyrolysis, partial combustion, or hydrothermal liquefaction.

In a particular embodiment, the thermal decomposition is gasification. The gasification is suitably conducted under the presence of a gasification agent such as oxygen, steam, carbon dioxide, or a combination thereof. Suitably also, the gasification agent is produced in the process; for instance, oxygen is provided by electrolysis and steam from the methanol conversion step.

As used herein, the term “suitably” is used interchangeably with the term “optionally”.

The raw syngas produced from the thermal decomposition, contrary to syngas produced from hydrocarbon feedstocks such as natural gas, is stoichiometric-insufficient on hydrogen. To compensate for the lack of hydrogen, at least a portion of the raw syngas is shifted to produce hydrogen according to the WGS reaction CO+H₂O→CO₂+H₂. Yet, since CO₂ is also produced, this excess CO₂ will then need to be removed to be still within the right methanol module M^{˜}2, where again M=(H₂−CO₂)/(CO₂+CO). The present invention removes the associated CO₂ emissions from the WGS, enabling additional methanol production by introducing renewable hydrogen. At least a portion of the raw syngas from the thermal decomposition is shifted via the WGS, further the CO/CO₂ molar ratio is adjusted to above 2, and hydrogen from electrolysis of a water feedstock, e.g. renewable hydrogen, is added, so the appropriate methanol module M^{˜}2 is still provided. Thereby, there is no need for an acid gas removal system producing CO₂, thus reducing or eliminating the attendant CO₂-emissions i.e. there is a significant lower carbon intensity (CI) of the process/plant, while at the same time producing a methanol synthesis gas that is provided with the right module and with a high reactivity: CO₂ emissions are drastically reduced or eliminated, as the available CO₂ is instead utilized for enabling the increase in the overall production of methanol by up to 40%. Other benefits in terms of less water production and reduce catalyst volume in the methanol synthesis reactor, as recited farther above, are also achieved.

In an embodiment, the renewable feed stream is a solid carbonaceous feed.

In a particular embodiment, the thermal decomposition is gasification which is optionally conducted in a plasma gasifier, and the renewable feed stream is refused derived fuel (RDF).

Suitably, there is also provided a water removal step for providing the RDF which is fed to the plasma gasifier.

Suitably also, there is also provided a water removal step for providing the renewable feed which is introduced to the thermal decomposition step.

As is well-known in the art, in a plasma gasifier, electrical energy is supplied for reaching a plasma torch, which gasifies the organic material of the renewable feed. y organic materials. Oxygen and/or air, suitably also steam, is also supplied to the plasma gasifier to provide the presence of oxygen which reacts with the carbon of the organic material, thereby producing the crude synthesis gas stream comprising carbon oxides (CO, CO₂) and hydrogen.

The term “refused derived fuel (RDF)” means a fuel produced from various types of waste, such as municipal solid waste (MSW), industrial waste or commercial waste. In accordance with the definition provided by Wikipedia.org as of 25 Apr. 2022, RDF consists largely of combustible components of such waste, as non-recyclable plastics (not including PVC), paper cardboard, labels, and other corrugated materials. These fractions are separated by different processing steps, such as screening, air classification, ballistic separation, separation of ferrous and non-ferrous materials, glass, stones and other foreign materials and shredding into a uniform grain size, or also pelletized in order to produce a homogeneous material which can be used as substitute for fossil fuels in e.g. cement plants, lime plants, coal fired power plants or as reduction agent in steel furnaces.

The crude synthesis gas stream is subsequently cleaned in e.g. the syngas purification section, whereby under the addition of e.g. water, the crude synthesis gas is depleted from impurities which may be detrimental for downstream steps, as explained farther above.

In an embodiment, in step c) the electrolysis is conducted in: an alkaline and/or polymer electrolyte membrane (PEM) electrolysis unit; or a solid oxide electrolysis cell unit (SOEC unit).

Alkaline and/or PEM electrolysis units; and solid oxide electrolysis cell units (SOEC units), are well-known in the art.

It would be understood, that liquid water cannot be passed through an SOEC unit, while steam cannot be passed through an alkaline and/or/PEM electrolysis unit. Hence, a SOEC unit operates with steam, while an alkaline and/or PEM unit operates with liquid water SOEC units may be operated at high temperatures, such as 700-800° C., which provides advantages over alkaline and/or PEM electrolysis units, which operate at much lower temperature, i.e. in the range 60-160° C. Such advantages include lower operational expenses due to lower cell voltage as well as lower capital expenses to higher current densities. Furthermore, when using SOEC for electrolysis of a water feedstock into H₂, thus based on steam, the energy for distillation of H₂O out of the produced methanol is saved.

In an embodiment, in step c) the electrolysis is conducted in a solid oxide electrolysis cell unit (SOEC unit), steam is generated in step e) i.e. the methanol conversion step, and the water feedstock for the SOEC unit comprises at least a portion of the steam generated in step e).

This reduces the need for steam import including import of demineralized water (DMW), as steam available in the process is used for the electrolysis.

In an embodiment, in step e), i.e. the step of converting the synthesis gas into methanol, comprises passing the methanol synthesis gas through a methanol synthesis reactor under the presence of a catalyst for producing a raw methanol stream, said step optionally further comprising a distillation step of the raw methanol stream for producing a water stream and a separate methanol stream having at least 98 wt % methanol.

In an embodiment, the process is absent of a steam reforming step for producing the raw synthesis gas stream, such as steam methane reforming step in a steam methane reformer (SMR), also known as tubular reforming or tubular steam reforming, or such as autothermal reforming in an autothermal reformer (ATR); or a steam reforming step combining SMR and ATR.

Thereby it is avoided to produce a raw synthesis gas with a high content of methane, which is typical for e.g. ATR, and which would require a different solution with reforming some of the purge gases from the loop in e.g. ATR. For the methanol production downstream, methane is an inert so there is an efficiency loss associated with the generation of methane.

In a second aspect of the invention, there is also provided a plant, i.e. process plant, for carrying out the method of any of the above or below embodiments.

Accordingly, there is provided a plant for carrying out the process of any of the embodiments of the first aspect of the invention, the plant comprising:
a water gas shift (WGS) section arranged to receive a raw synthesis gas stream and to provide a shifted synthesis gas;
an electrolysis unit arranged to receive a water feedstock and to provide a separate hydrogen containing stream and a separate oxygen containing stream;
a mixing point arranged to introduce at least a portion of the separate hydrogen containing stream into the shifted synthesis gas, thereby producing a methanol synthesis gas; wherein the methanol synthesis gas has a module M=(H₂−CO₂)/(CO+CO₂) in the range 1.80-2.40, such as 1.95-2.10, and a molar ratio CO/CO₂ greater than 2, such as 10 or higher; and
e) a methanol synthesis section arranged to receive the methanol synthesis gas and convert the methanol synthesis gas into methanol;
optionally, a cleaning unit arranged upstream said WGS section, and arranged to receive a crude synthesis gas stream and provide said raw synthesis gas;
optionally, a thermal decomposition unit, such as gasifier, arranged upstream said WGS section or upstream said cleaning unit, and arranged to receive a renewable feed stream and provide said crude synthesis gas stream.

Any of the embodiments and associated effects of the first aspect (process) of the invention may be used with the second aspect (plant) of the invention, or vice versa.

Advantages (benefits) of the invention include:
No CO₂ emissions
No need for expensive CO₂ removal technology
CO₂ is rather utilized for increasing the overall production of methanol by up to 40%
When applying SOEC for electrolysis based on steam, the energy for distillation of water out of the methanol being produced is saved
A simpler process and plant layout, thus reducing CAPEX and OPEX.

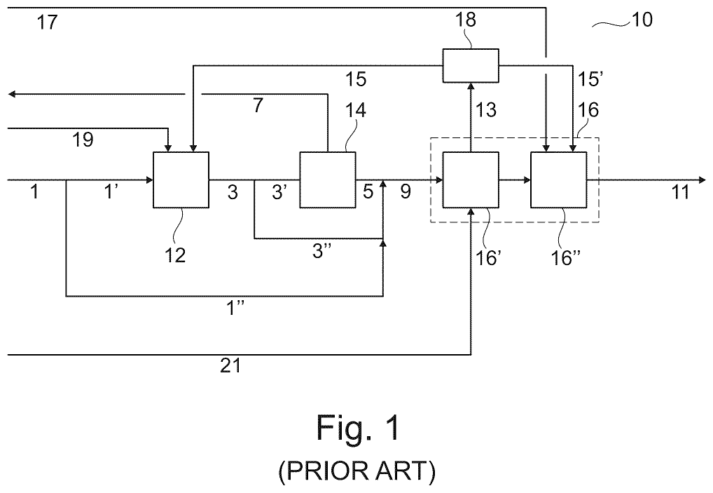

FIG. 1 shows a schematic process and plant layout in accordance with the prior art.

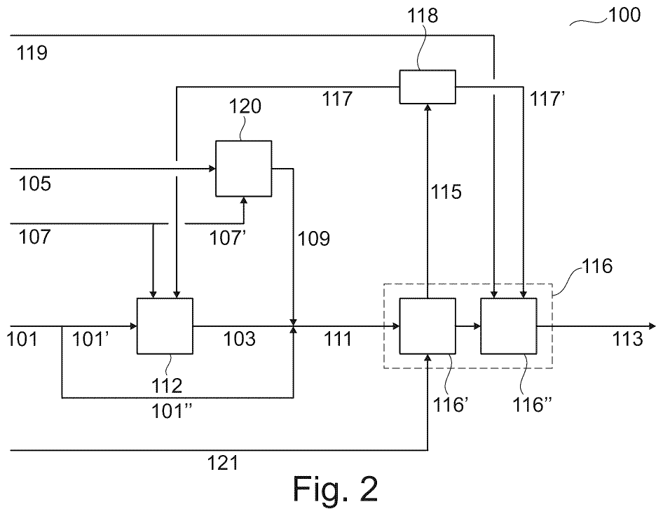

FIG. 2 shows a schematic process and plant layout in accordance with an embodiment of the present invention.

With reference to FIG. 1, a schematic layout 10 in accordance with the prior art is shown, in which a raw synthesis gas 1 is used to produce a methanol product 11. A portion 1′ of the raw synthesis gas is conducted to a water gas shift (WGS) step in WGS section 12 under the addition of water 19 by steam import, thereby producing shifted synthesis gas 3, 3′.3″. A portion 3′ of the shifted synthesis gas is conducted to a step of acid gas removal, typically CO₂-removal in CO₂-removal section 14, thereby producing a CO₂-stream 7 which is vented to the atmosphere, as well as a shifted and CO₂-depleted synthesis gas 5. A portion 1″ of the raw synthesis gas bypasses the WGS step and is combined with shifted synthesis gas 3″ which bypasses the CO₂-removal step, and which is then combined with the shifted and CO₂-depleted synthesis gas 5 to produce methanol synthesis gas 9. This syngas 9 is then conducted to a methanol conversion step in a methanol synthesis section 16 comprising methanol synthesis loop 16′ (methanol loop) including a methanol synthesis reactor (not shown) for producing a raw methanol stream, and methanol distillation section 16″ thereby producing methanol product 11, suitably with a purity of 98 wt % methanol or higher. Steam 13 produced in the methanol loop 16′ may be directed to a steam generation section 18, which thus generates steam 15, 15′ used in the WGS section 12 and methanol distillation section 16″. Boiler feed water 21 is suitably also added to the methanol loop 16′.

Now with reference to FIG. 2, a schematic layout 100 in accordance with an embodiment of the present invention is shown. A raw synthesis gas 101, suitably produced in a prior thermal decomposition step in thermal decomposition unit such as a gasification unit being fed with a renewable feed stream, is used to produce methanol product 113. A portion 101′ of the raw synthesis gas is conducted to a water gas shift (WGS) step in WGS section 112 under the addition of water 107 by steam import, thereby producing shifted synthesis gas 103. There is no acid gas removal, such as CO₂-removal in a downstream CO₂-removal section. A portion 101″ of the raw synthesis gas bypasses the WGS step and is combined with shifted synthesis gas 103, together with a separate hydrogen containing stream 109, thereby producing methanol synthesis gas 111. The hydrogen containing stream 109 is prepared by electrolysis of steam 105, 107′ in solid oxide electrolysis cell unit (SOEC unit) 120. The stream 105 is for instance demineralized water (DMW). The methanol synthesis gas 111 is then conducted, as in connection with FIG. 1, to a methanol conversion step in a methanol synthesis section 116 comprising methanol synthesis loop 116′ (methanol loop) including a methanol synthesis reactor (not shown) for producing a raw methanol stream, and methanol distillation section 116″ thereby producing e-methanol as the methanol product 113, suitably with a purity of 98 wt % methanol or higher. Steam 115 produced in the methanol loop 116′ may be directed to a steam production section 118, which thus generates steam 117, 117′ used in the WGS section 112 and methanol distillation section 116″. Boiler feed water 121 is suitably also added to the methanol loop 116′.

### EXAMPLE

The table below shows a comparison of the process/plant scheme according to FIG. 1 (prior art) vs the process/plant scheme according to FIG. 2 (embodiment of the invention). By the invention there is more than 40% additional methanol production while at the same time eliminating the amount of carbon dioxide vented to the atmosphere (CO₂ vent) from about 10000 tons per year, to zero:

| | Stream no. | Stream no. | FIG. 1 | FIG. 2 |
|---|---|---|---|---|
| | FIG. 1 | FIG. 2 | (Prior art) | (Invention) |
| Methanol syngas | 9 | 111 | 4450 | 4450 |
| (Nm³/h) | | | | |
| Hydrogen (Nm³/h) | — | 109 | 0 | 1620 |
| Steam: | | | | |
| Medium pressure | 19 | 107 | 1030 | 606 |
| (MP) steam import | | | | |
| (kg/h) | | | | |
| Low pressure (LP) | 17 | 119 | 759 | 1861 |
| steam import | | | | |
| (kg/h) | | | | |
| CO₂ vent (Nm³/h) | | | 604 | 0 |
| CO₂ vent (tons per | | | 9894 | 0 |
| year)* | | | | |
| Methanol: (kg/h) | | | 1748 | 2487 |
| Methanol (MTPD) | | | 42.0 | 59.7 |
| Additional | | | 0 | 42 |
| methanol | | | | |
| production (%) | | | | |

*Assuming 365 days/year with a carbon dioxide density of 1.87 kg/m³

1. A process for producing methanol, comprising the steps of:
a) providing a raw synthesis gas stream;
b) water gas shifting (WGS) at least a portion of the raw synthesis gas stream, thereby producing a shifted synthesis gas;
c) preparing a separate hydrogen containing stream and a separate oxygen containing stream by electrolysis of a water feedstock;
d) introducing at least a portion of the separate hydrogen containing stream into the shifted synthesis gas, thereby producing a methanol synthesis gas, wherein the methanol synthesis gas has a module M=(H₂−CO₂)/(CO+CO₂) in the range 1.80-2.40, and a molar ratio CO/CO₂ greater than 2; and
e) converting the methanol synthesis gas into said methanol.

2. The process according to claim 1, wherein the module M=(H₂−CO₂)/(CO+CO₂) is in the range 1.95-2.10, and the molar ratio CO/CO₂ is 10 or higher.

3. The process according to claim 1, wherein a portion of the raw synthesis gas bypasses the water gas shifting and is then combined with the shifted synthesis gas and the at least a portion of the separate hydrogen containing stream.

4. The process according to claim 1, wherein the process further comprises a cleaning step for providing the raw synthesis gas.

5. The process according to claim 4, wherein the process further comprises, prior to said step a), a thermal decomposition of a renewable feed stream for producing a crude synthesis gas stream, and subsequently subjecting the crude synthesis gas stream to said cleaning step for removing impurities, thereby producing the raw synthesis gas stream.

6. The process according to claim 5, wherein the thermal decomposition is gasification which is optionally conducted in a plasma gasifier, and the renewable feed stream is refused derived fuel (RDF).

7. The process according to claim 1, wherein in said step c) the electrolysis is conducted in: an alkaline and/or polymer electrolyte membrane electrolysis unit; or a solid oxide electrolysis cell unit (SOEC unit).

8. The process according to claim 1, wherein:
in said step c) the electrolysis is conducted in a solid oxide electrolysis cell unit (SOEC unit),
steam is generated in said step e) i.e. the methanol conversion step, and
the water feedstock for the SOEC unit comprises at least a portion of the steam generated in said step e).

9. The process according to claim 1, wherein the process is free of any steam reforming step for producing the raw synthesis gas stream.

10. A plant for carrying out the process of claim 1, the plant comprising:
a water gas shift (WGS) section configured to receive a raw synthesis gas stream and to provide a shifted synthesis gas;
an electrolysis unit configured to receive a water feedstock and to provide a separate hydrogen containing stream and a separate oxygen containing stream;
a mixing point configured to introduce at least a portion of the separate hydrogen containing stream into the shifted synthesis gas, thereby producing a methanol synthesis gas; wherein the methanol synthesis gas has a module M=(H₂−CO₂)/(CO+CO₂) in the range 1.80-2.40, and a molar ratio CO/CO₂ greater than 2; and
a methanol synthesis section configured to receive the methanol synthesis gas and convert the methanol synthesis gas into methanol;
optionally, a cleaning unit arranged upstream said WGS section, and configured to receive a crude synthesis gas stream and provide said raw synthesis gas;
optionally, a thermal decomposition unit arranged upstream said WGS section or upstream said cleaning unit, and configured to receive a renewable feed stream and provide said crude synthesis gas stream.

11. The process according to claim 1, wherein the process is free of any CO₂-removal step after said step b).

12. The plant according to claim 10, wherein the plant is free of any CO₂-removal section downstream of the water gas shift (WGS) section.
